(19) United States
(12) Patent Application Publication
(10) Pub. No.: US 2026/0183756 A1
(43) Pub. Date: Jul. 2, 2026

(54) CATALYST, METHOD FOR PRODUCING THE SAME, AND METHOD FOR PRODUCING ACRYLONITRILE

(71) Applicant: ASAHI KASEI KABUSHIKI KAISHA, Tokyo (JP)

(72) Inventors: Atsushi TOMODA, Tokyo (JP); Shota AIKI, Tokyo (JP)

(73) Assignee: ASAHI KASEI KABUSHIKI KAISHA, Tokyo (JP)

(21) Appl. No.: 18/837,343

(22) PCT Filed: Feb. 22, 2023
(86) PCT No.: PCT/JP2023/006547

§ 371 Date: Aug. 9, 2024

(30) Foreign Application Priority Data
Feb. 25, 2022 (JP) 2022-027850
Aug. 30, 2022 (JP) 2022-137016

(51) Int. Cl. B01J 23/887; B01J 21/08; B01J 23/00; B01J 35/61; B01J 35/63; B01J 35/69; B01J 37/02; B01J 37/04; C07C 253/26

(52) U.S. Cl. CPC B01J 23/8876; B01J 21/08; B01J 23/002; B01J 35/613; B01J 35/633; B01J 35/69; B01J 37/0236; B01J 37/04; C07C 253/26; B01J 2523/14; B01J 2523/22; B01J 2523/3712; B01J 2523/54; B01J 2523/68; B01J 2523/842; B01J 2523/845; B01J 2523/847

(57) ABSTRACT

A catalyst including a metal oxide, which is an oxide of a metal including molybdenum, bismuth and iron, and silica supporting the metal oxide, wherein when a surface parameter of silicon by SEM-EDX is A and a bulk parameter of silicon by XRF is B, A/B is 0.95 to 1.04.

### TECHNICAL FIELD

The present invention relates to a catalyst, a method for producing the same and a method for producing acrylonitrile.

### BACKGROUND ART

A known method for producing acrylonitrile is the reaction of propylene with ammonia and oxygen in the presence of a catalyst (ammo-oxidation reaction). Various catalysts for ammo-oxidation reaction and their production methods have been reported.

For example, Patent Literature 1 discloses “a catalyst comprising molybdenum, bismuth, iron, and nickel, wherein a proportion of a surface concentration of the nickel to a bulk concentration of the nickel is 0.60 to 1.20”.

Patent Literature 2 discloses “a catalyst for acrylonitrile synthesis, which contains a composite body of a metal oxide and a silica that supports the metal oxide, wherein the ratio of the specific surface area B of the catalyst for acrylonitrile synthesis after the acceleration test described below to the specific surface area A of the catalyst for acrylonitrile synthesis is 0% or more and 60% or less, the ratio being expressed by 100×(A−B)/A. (Acceleration test): The catalyst for acrylonitrile synthesis is heated in the air at 650° C. for 10 hours”.

Patent Literature 3 discloses “a method for producing an ammoxidation catalyst, comprising: a step of preparing a precursor slurry that is a precursor of the catalyst; a drying step of obtaining a dry particle from the precursor slurry; and a calcination step of calcining the dry particle, wherein the step of preparing the precursor slurry is a step of mixing a first solution or slurry having a first pH and a second solution or slurry to obtain a solution or slurry having a second pH after completion of mixing, a time during which a pH of a mixture passes through a particular range having an upper limit and a lower limit while the second solution or slurry is mixed is 1-70 seconds, the upper limit and the lower limit being designated as a third pH and a fourth pH respectively, and the third pH and the fourth pH are set between the first pH and the second pH”.

Patent Literature 4 discloses “a catalyst for acrylonitrile synthesis which is composed of a composite oxide including at least molybdenum and particles containing silica, wherein when the Mo/Si atomic ratio in bulk composition of the catalyst is represented by A and the Mo/Si atomic ratio in surface composition of the particles is represented by B, B/A is not more than 0.45”.

### CITATION LIST

### Patent Literature

Patent Literature 1: International Publication No. 2020/213361
Patent Literature 2: International Publication No. 2020/184330
Patent Literature 3: International Publication No. 2017/188349
Patent Literature 4: Japanese Patent No. 4242197

### SUMMARY OF INVENTION

### Technical Problem

Various catalysts for producing acrylonitrile by ammo-oxidation reaction have been reported, but there is still room for improvement. Therefore, an object of the present invention is to provide a catalyst suitable for the production of acrylonitrile, a method for producing the same, and a method for producing acrylonitrile using the catalyst.

### Solution to Problem

The present inventors have conducted intensive studies and have found that a catalyst suitable for the production of acrylonitrile can be produced by appropriately controlling the mass ratio of silicon on the surface of the catalyst and in the entire catalyst which includes a metal oxide and silica supporting the metal oxide.

The present invention includes the following embodiments.
[1]

A catalyst comprising a metal oxide, which is an oxide of a metal including molybdenum, bismuth and iron, and silica supporting the metal oxide,
wherein when a surface parameter of silicon by SEM-EDX is A and a bulk parameter of silicon by XRF is B, A/B is 0.95 to 1.04.
[2]

The catalyst according to [1], wherein a mass ratio of the silica to the metal oxide is 7:3 to 2:8.
[3]

The catalyst according to [1], wherein A/B is 0.95 to 1.02.
[4]

The catalyst according to any of [1] to [3], which is used for a vapor-phase catalytic ammoxidation reaction.
[5]

The catalyst according to any of [1] to [4], wherein the metal oxide is represented by the following formula (1):

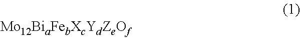

wherein
X is at least one selected from the group consisting of nickel, cobalt, magnesium, calcium, zinc, strontium and barium,
Y is at least one selected from the group consisting of cerium, chromium, lanthanum, neodymium, yttrium, praseodymium, samarium, gallium and indium,
Z is at least one selected from the group consisting of sodium, potassium, rubidium and cesium,
a is 0.1 or more and 2.0 or less,
b is 0.1 or more and 2.8 or less,
c is 0.1 or more and 10.0 or less,
d is 0.1 or more and 3.0 or less,
e is 0.01 or more and 2.0 or less, and
f is a value determined by X to Z and a to e.
[6]

The catalyst according to any of [1] to [5], wherein the catalyst has a specific surface area of 10 to 50 m²/g.
[7]

The catalyst according to any of [1] to [6], wherein
in a pore distribution of the catalyst,
the catalyst has a first modal diameter being within a range of 5 to 25 nm in a first range in which the pore diameter is 0 nm or more and less than 30 nm,
the catalyst has a second modal diameter being within a range of 35 to 200 nm in a second range in which the pore diameter is 30 nm or more and 1,000 nm or less,
the catalyst has a total volume of the pores of 0.05 cm³/g or more in the first range, and
the catalyst has a total volume of the pores of 0.05 cm³/g or more in the second range.
[8]

A method for producing a catalyst, comprising:
a step of providing a first mixture which comprises a silica starting material and molybdenum and has a pH of 4.1 to 5.5,
a step of preparing a second mixture by mixing the first mixture with bismuth and iron,
a step of preparing particles by spray-drying the second mixture, and
a step of preparing catalyst by calcining the particles.
[9]

The method according to [8], wherein the pH of the first mixture is adjusted to 4.1 to 5.5 by ammonia water or nitric acid.
[10]

The method according to [8] or [9], wherein the silica starting material is silica sol and the silica sol has a median diameter of 50 nm or less.
[11]

A method for producing acrylonitrile, comprising:
a step of preparing acrylonitrile by reacting propylene, ammonia and molecular oxygen in the presence of the catalyst according to any one of [1] to [7].
[12]

The method according to [11], wherein the reaction is performed in a fluidized bed reactor.

### Advantageous Effect of Invention

The present invention can provide a catalyst suitable for the production of acrylonitrile, a method for producing the same, and a method for producing acrylonitrile using the catalyst.

### DESCRIPTION OF EMBODIMENTS

Hereinafter embodiments of the present invention will be described in detail, but the present invention is not limited thereto and may be modified in various ways without departing from the gist of the present invention.

### <Catalyst>

An embodiment of the present invention relates to a catalyst comprising a metal oxide, which is an oxide of a metal including molybdenum, bismuth and iron, and silica supporting the metal oxide, wherein when a surface parameter of silicon by SEM-EDX is A and a bulk parameter of silicon by XRF is B, A/B is 0.95 to 1.04.

Surface parameter (A) of silicon by SEM-EDX is the ratio of the mass of silicon atoms to the total mass of atoms other than oxygen constituting the catalyst on the surface of the catalyst.

Bulk parameter (B) of silicon by XRF is the ratio of the mass of silicon atoms to the total mass of atoms other than oxygen constituting the catalyst in the entire catalyst.

The catalyst according to the present embodiment is suitable for producing acrylonitrile. For example, acrylonitrile can be produced in high yield with the catalyst according to the present embodiment, and the catalyst has excellent wear resistance.

The following is assumed to be the reason why acrylonitrile can be produced in high yield with the catalyst according to the present embodiment and the catalyst has excellent wear resistance, but the present invention is not limited in any way by the following assumed reason.

That is, when A/B is small, the amount of metal oxide on the surface of the catalyst relatively increases, and since the metal oxide has activity to convert propylene into acrylonitrile, the yield of acrylonitrile is improved due to the presence of a large amount of the metal oxide on the surface of the catalyst that comes into contact with propylene.

By contrast, when A/B is large, the amount of silica on the surface of the catalyst relatively increases, and since silica has high strength, wear resistance of the catalyst is improved due to the presence of a large amount of silica on the surface of the catalyst.

Since A/B is within the appropriate range in the catalyst according to the present embodiment, both the activity and the wear resistance of the catalyst seem to be achieved.

Surface parameter (A) of silicon by SEM-EDX is preferably 0.288 or more, more preferably 0.290 or more and further preferably 0.292 or more from the viewpoint of the improvement in the wear resistance of the catalyst.

Surface parameter (A) of silicon by SEM-EDX is preferably 0.325 or less, more preferably 0.310 or less, and further preferably 0.305 or less from the viewpoint of the improvement in the yield of acrylonitrile.

The range of surface parameter (A) of silicon by SEM-EDX may be determined by appropriately combining the lower limit and the upper limit described above. For example, the range of surface parameter (A) is preferably 0.288 to 0.325, more preferably 0.290 to 0.310, and further preferably 0.292 to 0.305.

### [Metal Oxide]

The metal oxide contained in the catalyst according to the present embodiment is a composite metal oxide including molybdenum (Mo), bismuth (Bi) and iron (Fe). The metal oxide may contain additional metal atoms. Examples of additional metal atoms include nickel (Ni), cobalt (Co), magnesium (Mg), calcium (Ca), zinc (Zn), strontium (Sr), barium (Ba), cerium (Ce), chromium (Cr), lanthanum (La), neodymium (Nd), yttrium (Y), praseodymium (Pr), samarium (Sm), gallium (Ga), indium (In), sodium (Na), potassium (K), rubidium (Rb) and cesium (Cs).

It is preferable that the metal oxide is represented by the following formula (1) from the viewpoint of further improvement in the yield of acrylonitrile.

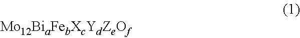

In the formula (1), X is preferably at least one selected from the group consisting of nickel, cobalt, magnesium, calcium, zinc, strontium and barium, more preferably at least one selected from the group consisting of nickel, cobalt and magnesium, and further preferably at least two selected from the group consisting of nickel, cobalt and magnesium. Specific examples of X include a combination of nickel and cobalt and a combination of nickel and magnesium.

In the formula (1), Y is preferably at least one selected from the group consisting of cerium, chromium, lanthanum, neodymium, yttrium, praseodymium, samarium, gallium and indium, and more preferably cerium.

In the formula (1), Z is preferably at least one selected from the group consisting of sodium, potassium, rubidium and cesium, and more preferably rubidium.

In the formula (1), a is preferably 0.1 or more and 2.0 or less, more preferably 0.2 or more and 1.0 or less, and further preferably 0.3 or more and 0.5 or less.

In the formula (1), b is preferably 0.1 or more and 2.8 or less, more preferably 0.5 or more and 2.5 or less, and further preferably 1.0 or more and 2.0 or less.

In the formula (1), c is preferably 0.1 or more and 10.0 or less, more preferably 3.0 or more and 9.5 or less, and further preferably 6.0 or more and 9.0 or less. When X includes two or more metal atoms, c is the sum of the metal atoms.

In the formula (1), d is 0.1 or more and 3.0 or less, more preferably 0.2 or more and 2.0 or less, and further preferably 0.3 or more and 1.5 or less. When Y includes two or more metal atoms, d is the sum of the metal atoms.

In the formula (1), e is preferably 0.01 or more and 2.0 or less, more preferably 0.03 or more and 1.0 or less, and further preferably 0.06 or more and 0.5 or less.

In the formula (1), f is a value determined by X to Z and a to e above, in other words, a value determined by the valence of other elements.

The composition of the metal oxide may be measured by the method described in Examples.

### [Support]

The metal oxide contained in the catalyst according to the present embodiment is supported on a support. The support is silica.

### [Catalyst]

The catalyst according to the present embodiment may be suitably used for vapor-phase catalytic ammoxidation reaction. The vapor-phase catalytic ammoxidation reaction is a reaction in which hydrocarbon, ammonia and molecular oxygen are allowed to react in a gas phase to obtain unsaturated nitrile.

In the catalyst according to the present embodiment, the ratio of the surface parameter of silicon by SEM-EDX to the bulk parameter of silicon by XRF (A/B) is 0.95 to 1.04, preferably 0.95 to 1.02, and more preferably 0.96 to 1.02. A/B of 0.95 or more tends to improve the wear resistance of the catalyst. A/B of 1.04 or less tends to improve the yield of acrylonitrile. The surface parameter of silicon by SEM-EDX and the bulk parameter of silicon by XRF may be measured by the method described in Examples.

The catalyst according to the present embodiment has a specific surface area of preferably 10 to 50 m²/g, more preferably 15 to 46 m²/g, and further preferably 20 to 42 m²/g. A specific surface area of 10 m²/g or more tends to provide high catalytic activity in the production of acrylonitrile. A specific surface area of 50 m²/g or less tends to reduce the exposure of active decomposition sites in propylene or acrylonitrile and increase the selectivity of acrylonitrile. The specific surface area may be measured by the method described in Examples.

In the pore distribution of the catalyst according to the present embodiment, the catalyst has a first modal diameter being within a range of preferably 5 to 25 nm, more preferably 10 to 20 nm, and further preferably 10 to 15 nm, in the first range in which the pore diameter is 0 nm or more and less than 30 nm. A first modal diameter being within a range of 5 to 25 nm is thought to allow the silica support to have a dense structure and thus improve the strength of the catalyst. The first modal diameter may be measured by the method described in Examples.

The catalyst has a total volume of the pores in the first range of preferably 0.05 cm³/g or more. The lower limit of 0.05 cm³/g is intended to eliminate noise in the pore distribution curve. The total volume of the pores in the first range may be measured by the method described in Examples.

In the pore distribution of the catalyst of the present invention, the catalyst has a second modal diameter being within a range of preferably 35 to 200 nm, more preferably 40 to 150 nm, and further preferably 45 to 100 nm in the second range in which the pore diameter is 30 nm or more and 1,000 or less. A second modal diameter being within a range of 35 to 200 nm is thought to allow the silica support and metal elements to align uniformly and thus improve the selectivity of acrylonitrile. The second modal diameter may be measured by the method described in Examples.

The catalyst has a total volume of the pores in the second range of preferably 0.05 cm³/g or more. The lower limit of 0.05 cm³/g is intended to eliminate noise in the pore distribution curve. The total volume of the pores in the second range may be measured by the method described in Examples.

The amount of the metal oxide supported in the catalyst according to the present embodiment is preferably 30 to 80% by mass, more preferably 40 to 70% by mass, and further preferably 50 to 65% by mass based on the mass of the catalyst.

The mass ratio between the support (silica) and the metal oxide in the catalyst according to the present embodiment is preferably 7:3 to 2:8, more preferably 6:4 to 3:7, and further preferably 5:5 to 3.5:6.5.

### <Method for Producing Catalyst>

An embodiment of the present invention relates to a method for producing a catalyst, comprising: a step of providing a first mixture which comprises a silica starting material and molybdenum and has a pH of 4.1 to 5.5, a step of preparing a second mixture by mixing the first mixture with bismuth and iron, a step of preparing particles by spray-drying the second mixture, and a step of preparing catalyst by calcining the particles.

The catalyst described in the section of <Catalyst> above may be produced by the method according to the present embodiment.

The following is assumed to be the reason why the method according to the present embodiment can produce a catalyst in which the ratio of the surface parameter of silicon by SEM-EDX to the bulk parameter of silicon by XRF (A/B) is 0.95 to 1.04, but the present invention is not limited in any way by the following assumed reasons.

That is, as the pH of the first mixture is reduced, the proportion of metal atoms dissolved is increased, and the amount of metal atoms that move to the surface of particles of the catalyst is increased along with evaporation of water in spray drying. This seems to increase the relative amount of the metal oxide on the surface of the catalyst, and thus reduce A/B.

Meanwhile, when the pH of the first mixture is increased, the proportion of metal atoms dissolved is reduced, and the amount of metal atoms that move to the surface of particles of the catalyst is decreased along with evaporation of water in spray drying. This seems to reduce the relative amount of the metal oxide on the surface of the catalyst, and thus increase A/B.

Since the pH of the first mixture is in an appropriate range in the method according to the present embodiment, A/B is assumed to be adjusted to the range of 0.95 to 1.04.

The method of preparing the first mixture is not limited. For example, the pH may be adjusted after making a mixture containing silica starting material and molybdenum, or molybdenum may be mixed after adjusting the pH of a liquid containing a silica starting material, or a silica starting material may be mixed after adjusting the pH of a liquid containing molybdenum.

Although the method is not limited, it is preferable that a liquid containing molybdenum adjusted to neutral to alkaline (e.g., pH 5.6 to 9.0) is added to the silica starting material, and then the pH is adjusted to 4.1 to 5.5 to prepare the first mixture. This method prevents precipitation of molybdenum.

The method of adjusting the pH of the first mixture is not limited, and for example, it is desired that the pH is adjusted by ammonia water or nitric acid from the viewpoint that ammonia water or nitric acid are composed of nitrogen, oxygen and hydrogen, and no metal element remains in the catalyst after calcination.

The first mixture has a pH of 4.1 to 5.5, preferably 4.1 to 5.2, and more preferably 4.1 to 4.8.

The silica starting material is not limited as long as it contains silica, and examples thereof include silica sol. The primary particle of silica dispersed in the silica sol has a median diameter of preferably 50 nm or less, and more preferably 10 to 50 nm.

The method of preparing the second mixture is not limited, and the first mixture may be mixed with bismuth and iron. Additional metal atoms may be mixed thereto to achieve the desired catalyst composition. Details of additional metal atoms and the catalyst composition are as described in the section of <Catalyst> above.

The second mixture has a pH of preferably 2.0 or less, more preferably 0 to 2.0, and further preferably 0 to 1.5.

The order of addition is not limited, and a carboxylic acid compound may be added thereto as needed before spray-drying the second mixture (in other words, the second mixture may include a carboxylic acid compound).

Since some precipitation forms when mixing the first mixture with bismuth and iron, it is preferable to add the carboxylic acid compound to the first mixture from the viewpoint of mixing of the carboxylic acid compound and the metal components.

Carboxylic acids are a typical organic coordination compound, and their binding to a metal component is thought to prevent aggregation of the metal component, allowing more metal components to be dissolved in the mixture, and increase the amount of metal moved to the surface of particles of the catalyst along with evaporation of water during spray drying. The carboxylic acid compound is not particularly limited, and a divalent or higher valent polycarboxylic acid is preferred, and examples thereof include oxalic acid, tartaric acid, succinic acid, malic acid and citric acid. Of them, oxalic acid and tartaric acid are preferred, and oxalic acid is more preferred.

The amount of carboxylic acid added is preferably 0.10 mole equivalent based on the total amount of the metal elements other than silicon in the second mixture from the viewpoint of inhibition of cracks in catalyst particles due to decomposition or diffusion of carboxylic acid in the process of calcination.

In the spray drying of the second mixture, the inlet temperature of the spray dryer is preferably 100 to 400° C., more preferably 150 to 350° C., and further preferably 200 to 300° C.

In the spray drying of the second mixture, the outlet temperature of the spray dryer is preferably 100 to 180° C., and more preferably 100 to 150° C.

The calcination temperature for particles obtained by spray drying is preferably 150 to 750° C., more preferably 300 to 700° C., and further preferably 500 to 650° C.

### <Method for Producing Acrylonitrile>

An embodiment of the present invention relates to a method for producing acrylonitrile, comprising a step of preparing acrylonitrile by reacting propylene, ammonia and molecular oxygen in the presence of the catalyst described in the section of <Catalyst> above.

The type of the reactor for performing the above reaction is not limited, and it is preferable to use a fluidized bed reactor.

In the above reaction, the molar ratio among propylene, ammonia and air is preferably 1.0:0.8 to 2.5:7.0 to 14.0, and more preferably 1.0:0.7 to 1.4:8.0 to 13.5

The reaction temperature is preferably 300 to 500° C., and more preferably 400 to 500° C.

The reaction pressure is preferably 0.01 to 0.5 MPa, and more preferably 0.05 to 0.3 MPa.

### EXAMPLES

Hereinafter the present invention will be described in detail with reference to Examples and Comparative Examples, but the technical scope of the present invention is not limited thereto.

### <Preparation of Catalyst>

### Example 1

Catalyst particles composed of 60% by mass of a composite metal oxide with a metal component composition represented by Mo_{12.00}Bi_{0.34} Fe_{1.58}CO_{4.38}Ni_{3.11}Ce_{0.89}Rb_{0.11} supported on 40% by mass of a support made of silica were prepared by the following procedure.

1066.7 g of an aqueous silica sol containing 30% by mass of the first silica was precisely weighed in such a manner that the support made of 40% by mass of silica included 80% by mass of the first silica with an average primary particle size of 12 nm and 20% by mass of the second silica with an average primary particle size of 41 nm. Next, 25.0 g of oxalic acid dihydrate dissolved in 287.5 g of water was added thereto, and 266.7 g of an aqueous silica sol containing 30% by mass of the second silica was also added thereto.

Subsequently, 36.0 g of a 15.0% by mass aqueous ammonia solution was added to a liquid prepared by dissolving 479.8 g of ammonium paramolybdate [(NH₄)₆Mo₇O₂₄·4H₂O] in 862.5 g of water, and the solution was mixed, and then was added to the above silica sol. 255.0 g of a 16.6% by mass nitric acid aqueous solution was also added thereto to give the first mixture. The pH of the first mixture was measured and the pH was 4.1.

Then, a solution prepared by dissolving 37.3 g of bismuth nitrate [Bi(NO₃)₃·5H₂O], 144.6 g of iron nitrate [Fe(NO₃)₃·9H₂O], 288.7 g of cobalt nitrate [Co(NO₃)₂·6H₂O], 204.8 g of nickel nitrate [Ni(NO₃)₂·6H₂O], 87.5 g of cerium nitrate [Ce(NO₃)₃·6H₂O] and 3.67 g of rubidium nitrate [RbNO₃] in 394.2 g of a nitric acid aqueous solution with a concentration of 16.6% by mass was added to the above first mixture to give the second mixture (starting material slurry).

Next, using a spraying device which was arranged at the center of the top of the dryer and equipped with a dish-shaped rotor, the second mixture was spray-dried under conditions of an inlet temperature of about 230° C. and an outlet temperature of about 110° C. The rotational speed of the disk was set to 12,500 rpm. The resulting dried body (particles) was kept at 200° C. for 5 minutes, and the temperature was increased from 200° C. to 450° C. at 2.5° C./minute and kept at 450° C. for 20 minutes to denitrate. The resulting denitrated powder was calcined at 600° C. for 2 hours to give catalyst particles.

### Examples 2 to 10 and Comparative Examples 1 to 7

Catalyst particles were prepared in the same manner as in Example 1 under conditions shown in Table 1-1, Table 2-1 and Table 3-1.

### Example 11

Catalyst particles composed of 60% by mass of a composite metal oxide with a metal component composition represented by Mo_{12.00}Bi_{0.34}Fe_{1.48}Ni_{6.35} Mg_{1.70}Ce_{0.53}Rb_{0.17} supported on 40% by mass of a support made of silica were prepared by the following procedure.

1066.7 g of an aqueous silica sol containing 30% by mass of the first silica in such a manner that the support made of 40% by mass of silica included 80% by mass of the first silica with an average primary particle size of 12 nm and 20% by mass of the second silica with an average primary particle size of 41 nm was mixed with 266.7 g of an aqueous silica sol containing 30% by mass or the second silica.

Subsequently, 36.0 g of a 15.0% by mass aqueous ammonia solution was added to a liquid prepared by dissolving 494.6 g of ammonium paramolybdate [(NH₄)₆Mo₇O₂₄·4H₂O] in 889.0 g of water, and the solution was mixed, and then was added to the above silica sol. 250.0 g of a 16.6% by mass nitric acid aqueous solution was also added thereto to give the first mixture. The pH of the first mixture was measured and the pH was 4.2.

Then, a solution prepared by dissolving 38.5 g of bismuth nitrate [Bi(NO₃)₃·5H₂O], 139.6 g of iron nitrate [Fe(NO₃)₃·9H₂O], 431.1 g of nickel nitrate [Ni(NO₃)₂·6H₂O], 101.7 g of magnesium nitrate [Mg(NO₃)₂·6H₂O], 53.7 g of cerium nitrate [Ce(NO₃)₃·6H₂O] and 5.85 g of rubidium nitrate [RbNO₃] in 399.5 g of a nitric acid aqueous solution with a concentration of 16.6% by mass was added to the above first mixture to give the second mixture (starting material slurry).

Next, using a spraying device which was arranged at the center of the top of the dryer and equipped with a dish-shaped rotor, the second mixture was spray-dried under conditions of an inlet temperature of about 230° C. and an outlet temperature of about 110° C. The rotational speed of the disk was set to 12, 500 rpm. The resulting dried body (particles) was kept at 200° C. for 5 minutes, and the temperature was increased from 200° C. to 450° C. at 2.5° C./minute and kept at 450° C. for 20 minutes to denitrate. The resulting denitrated powder was calcined at 580° C. for 2 hours to give catalyst particles.

### Examples 12 to 14 and Comparative Examples 8 to 10

Catalyst particles were prepared in the same manner as in Example 11 under conditions shown in Table 4-1.

### <Measurement of A/B and Composition of Metal Oxide>

Ratio A of the mass of silicon atoms to the total mass of atoms other than oxygen constituting the catalyst on the surface of the catalyst, ratio B of the mass of silicon atoms to the total mass of atoms other than oxygen constituting the catalyst in the entire catalyst, and the composition ratio of the metal oxide represented by formula (1) were calculated by the following method. The results are as shown in Tables 1-2 to 4-2.

### [Method for Calculating A]

The surface of the catalyst was measured by energy dispersive X-ray spectrometry (EDX) using an electron microscope (SEM: scanning electron microscopy) to calculate surface parameter (A) of silicon by SEM-EDX.

As the surface parameter of the catalyst is calculated in the measurement using SEM-EDX, the parameter refers to the ratio of silicon at a thickness about 2 μm from the outer surface.

The specific procedure of the measurement will be described in the following.

In the pretreatment of the measurement, a 10 mm square carbon tape was stretched over a φ 15 mm carbon sample stand, and the catalyst particles were laid on the tape and fixed. Then the surface of the catalyst particles was coated with osmium tetroxide to form a metallic osmium layer, and the particles were subjected to conductive treatment and measurement. For coating of osmium tetroxide, osmic acid (manufactured by Nisshin EM Co., Ltd.) and Osmium Coater HPC-1SW manufactured by Vacuum Device Inc. were used, and the time of coating was 5 seconds.

SU-70 equipped with a Schottky electron gun manufactured by Hitachi, Ltd. was used as SEM. EMAX X-max50 manufactured by HORIBA, Ltd. was used as an EDX detector. The area of the element of the detector was 50 mm² and the angle of the detector was 30°.

The accelerating voltage of SEM was set to 15 kV, the emission current was 26 μA and the working distance from the objective lens was 15 mm. The analytical area for EDX was a rectangular area of 300 μm×400 μm, observed at a magnification of 300. The area of observation was set so as to include about 20 catalyst particles. The collection time of the spectrum in the measurement was 300 seconds, and the process time was set to 3. The range of the spectrum was 0 to 20 keV.

The following excitation beams: Mo-L beam, Bi-M beam, Fe—K beam, Ni—K beam, Co—K beam, Mg—K beam, Ce-L beam, and Si—K beam were used to quantify atoms. When measuring other atoms, excitation beams suitable for quantitation are used as needed.

Peak intensities were obtained as the integrated area of the peaks, and quantitative values of the concentration of atoms other than oxygen and carbon constituting the catalyst were determined by the XPP method using analysis software INCA manufactured by Oxford Instruments. The surface parameter of silicon by SEMEDX was calculated. The X-ray intensity of the respective atoms used in the quantitative calculation was obtained by removing the continuous X-ray component and, in the case of peak overlap, the X-ray intensity was obtained by peak separation. If the amount of metal contained was so small that the peak was not detectable or could not be separated, the metal could not be quantified and thus was excluded from the calculation. For the XPP method, Quantitative Analysis of Homogeneous or Stratified Microvolumes Applying the Model “PAP” Electron Probe Quantitation pp 31-75 Jean-Louis PouchouFrancoise Pichoir (1991) was used as a reference.

### [Method for Calculating B]

The catalyst was measured by X-ray fluorescence (XRF) spectrometer to calculate bulk parameter (B) of silicon by XRF.

First, 4.0 g of the catalyst particles and 10 balls of SUS304 with a diameter of 10 mm were placed in a container made of SUS304 having a capacity of 80 ml. The particles were pulverized using planetary ball mill LP-4 manufactured by Ito Seisakusho Co., Ltd at 175 rpm for 30 minutes.

3.4 g of the pulverized catalyst powder was precisely weighed and placed in the inside of a polyvinyl chloride ring with an outer diameter of 37 mm, an inner diameter of 30 mm and a thickness of 5 mm, and pressurized at 25 tf for 30 seconds using an M-type hydraulic compression tester manufactured by MAEKAWA TESTING MACHINE MFG. Co., Ltd. to give a compression-molded sample.

The quantitative value of the concentration of atoms in the resulting sample was determined by the fundamental parameter method using a scanning X-ray fluorescence spectrometer ZSX Primus III+ manufactured by Rigaku Corporation to calculate the bulk parameter of silicon by XRF. In this case, Rh was used as the target material for the X-ray tube, the output power was 3 kW, and LiF, PET, RX26, Ge, RX45, RX61, and RX75 were used as the analyzing crystal. Suitable target materials and analyzing crystals were used depending on the elements contained in the composition.

### [Method of Calculating Composition of Metal Oxide Represented by Formula (1)]

The quantitative value of the concentration of atoms in the sample was determined under the same measurement conditions as for the bulk parameter of silicon by XRF to calculate the composition of the metal oxide represented by formula (1).

### <Reaction Conditions and Yield in the Production of Acrylonitrile by Ammoxidation Reaction of Propylene>

Acrylonitrile and hydrogen cyanide were produced by ammoxidation reaction of propylene using each of the catalysts prepared in Examples and Comparative Examples. The reaction tube used in this process was a Pyrex (registered trademark) glass tube with an inner diameter of 25 mm that incorporated 16 pieces of 10-mesh wire mesh at 1 cm intervals.

The amount of the catalyst was set to 50 cc, the reaction temperature was set to 430° C. and the reaction pressure was set to 0.17 MPa, and the reaction was performed by supplying a mixed gas of propylene/ammonia/air at a total gas flow rate of 250 to 450 cc/sec (in terms of NTP). At that stage, the content of propylene in the mixed gas was 9% by volume, and the molar ratio of propylene/ammonia/air was 1/(0.7 to 1.4)/(8.0 to 13.5). Within that range the flow rate of ammonia was changed so that the sulfuric acid basic unit defined by the following equation was 20±2 kg/T-AN, and the flow rate of air was changed so that the oxygen concentration in the gas at the outlet of the reactor was 0.2±0.02% by volume. The molar ratio of ammonia/propylene in that case was defined as N/C. Furthermore, by changing the total flow rate of the mixed gas, the contact time defined by the following equation was changed and set so that the conversion ratio of propylene defined by the following equation was 99.3±0.2%.

The yield of acrylonitrile produced by the reaction was as a value defined by the following equation. The results are as shown in Tables 1-2 to 4-2.

$$Sulfuric acid unit ( kg / T - AN ) = \frac{Weight of sulfuric acid required to neutralize unreacted ammonia ( kg )}{Weight of acrylonitrile produced ( T )}$$

$$Contact time ( sec . ) = \frac{Amount of catalyst ( cc )}{Flow rate of mixed gas ( cc - NTP / sec )} \times \frac{273}{273 + Reaction temperature ( °C )} \times \frac{Reaction pressure ( MPa )}{0.1}$$

$$Conversion ratio of propylene ( \% ) = \frac{Propylene consumed ( moles )}{Propylene supplied ( moles )} \times 100$$

$$Yield of acrylonitrile ( \% ) = \frac{Acrylonitrile produced ( moles )}{Propylene supplied ( moles )} \times 100$$

### <Measurement of Wear Resistant Strength (ATT Strength)>

The wear resistant strength (attrition strength) of the catalyst was measured as wear loss according to the method described in “Test Method for Synthetic Fluid Cracking Catalyst” (American Cyanamid Co. Ltd. 6/31-4m-1/57) (hereinafter referred to as the “ACC method”).

The attrition strength was evaluated in terms of wear loss, and wear loss was defined as follows.

$$Wear loss ( \% ) = R / ( S\_Q ) \times 100$$

In the above equation, Q is the mass of the catalyst (g) worn and scattered to the outside in 0 to 5 hours and R is the mass of the catalyst (g) worn and scattered to the outside in 5 to 20 hours. S is the mass of the catalyst (g) subjected to the test.

Catalysts with a value of wear loss of 1.5% or less were judged to be applicable for long-term stable industrial use. The results are as shown in Tables 1-2 to 4-2.

### <Measurement of Specific Surface Area>

The specific surface area of the catalysts was measured using an automatic specific surface area analyzer, Gemini VII manufactured by Micromeritics Instruments Corporation, using the following procedure.

0.5 g of each of the catalysts was placed in a standard cell, and the standard cell was installed on a deaerator Flow Prep 060 to pre-dry the catalyst under helium at 300° C. for 15 minutes. After pre-drying, the specific surface area of the catalyst was measured by the one-point BET method using nitrogen as adsorption gas. The results are as shown in Tables 1-2 to 4-2.

### <Measurement of Pore Diameter and Pore Volume>

The pore distribution of the catalysts was measured by the following method. Two types of measurement devices appropriate for the pore diameter ranges to be measured were used.

[Measurement for Pore Diameter Range of 0 mm or More and Less than 30 nm]

The pore distribution of the catalysts was measured using an automatic pore distribution analyzer, 3-flex, manufactured by Micromeritics Instruments Corporation at liquid nitrogen temperature.

0.5 g of each of the catalysts was placed in the standard cell, and the standard cell was installed on the deaerator Flow Prep 060 to pre-dry the catalyst under reduced pressure at 250° C. for 18 hours. For the sample after the treatment, the pore distribution of the catalyst was measured based on the desorption isotherm curve by the BJH method using nitrogen as an adsorption gas, and the modal diameter of the pore diameters and values of the pore volume per 1 g were obtained. The results are as shown in Tables 1-2 to 4-2.

### [Measurement for Pore Diameter Range of 30 mm or More and 1,000 nm or Less]

The pore distribution of the catalysts was measured using a mercury porosimeter AutoPore 9500 manufactured by Micromeritics Instruments Corporation at room temperature of about 25° C.

The amount of sample was 0.75 g, and the pore distribution of 30 nm or more and 1,000 or less was measured at a measurement pressure of 30 to 33,000 psia, and the modal diameter of the pore diameters and values of the pore volume per 1 g were obtained. The results are as shown in Tables 1-2 to 4-2.

TABLE 1-1

| | | | | | | | | | | | Amount | Amount of | | Calcination |
|---|---|---|---|---|---|---|---|---|---|---|---|---|---|---|
| | | | | | | | | | 12 nm | 41 nm | of oxalic | 16.6 wt % | First | temperature |
| | | | | | | | | | silica | silica | acid | nitric acid | mixture | for catalyst |
| | Mo | Bi | Fe | Co | Ni | Mg | Ce | Rb | sol wt % | sol wt % | added (g) | added (g) | pH | (° C.) |
| Example 1 | 12.00 | 0.34 | 1.58 | 4.38 | 3.11 | 0 | 0.89 | 0.11 | 80 | 20 | 25 | 255 | 4.1 | 600 |
| Example 2 | 12.00 | 0.34 | 1.58 | 4.38 | 3.11 | 0 | 0.89 | 0.11 | 80 | 20 | 25 | 194 | 4.4 | 600 |
| Example 3 | 12.00 | 0.34 | 1.58 | 4.38 | 3.11 | 0 | 0.89 | 0.11 | 80 | 20 | 25 | 162 | 4.7 | 600 |
| Example 4 | 12.00 | 0.34 | 1.58 | 4.38 | 3.11 | 0 | 0.89 | 0.11 | 80 | 20 | 0 | 255 | 4.3 | 600 |
| Comparative | 12.00 | 0.34 | 1.58 | 4.38 | 3.11 | 0 | 0.89 | 0.11 | 80 | 20 | 25 | 296 | 3.9 | 600 |
| Example 1 | | | | | | | | | | | | | | |
| Comparative | 12.00 | 0.34 | 1.58 | 4.38 | 3.11 | 0 | 0.89 | 0.11 | 80 | 20 | 25 | 0 | 5.7 | 600 |
| Example 2 | | | | | | | | | | | | | | |
| Comparative | 12.00 | 0.34 | 1.58 | 4.38 | 3.11 | 0 | 0.89 | 0.11 | 80 | 20 | 0 | 296 | 4.0 | 600 |
| Example 3 | | | | | | | | | | | | | | |

TABLE 1-2

| | | | | | | Specific | 0 to 30 | 0 to 30 | 30 to 1,000 | 30 to 1,000 |
|---|---|---|---|---|---|---|---|---|---|---|
| | A: Si atom | B: Si atom | | AN | Wear | surface | nm modal | nm pore | nm modal | nm pore |
| | on particle | in entire | Ratio | yield | loss | area | diameter | volume | diameter | volume |
| | surface wt % | particle wt % | A/B | (%) | (%) | (m²/g) | (nm) | (ml/g) | (nm) | (ml/g) |
| Example 1 | 0.293 | 0.305 | 0.962 | 85.1 | 0.6 | 36 | 11 | 0.12 | 59 | 0.07 |
| Example 2 | 0.301 | 0.305 | 0.988 | 85.0 | 0.6 | 36 | 11 | 0.12 | 57 | 0.08 |
| Example 3 | 0.308 | 0.305 | 1.012 | 84.9 | 0.5 | 35 | 11 | 0.12 | 57 | 0.07 |
| Example 4 | 0.312 | 0.305 | 1.026 | 84.8 | 0.4 | 35 | 11 | 0.11 | 57 | 0.07 |
| Comparative | 0.277 | 0.305 | 0.910 | 84.1 | 1.9 | 35 | 10 | 0.11 | 58 | 0.08 |
| Example 1 | | | | | | | | | | |
| Comparative | 0.342 | 0.305 | 1.122 | 84.6 | 0.3 | 35 | 10 | 0.12 | 51 | 0.07 |
| Example 2 | | | | | | | | | | |
| Comparative | 0.287 | 0.305 | 0.942 | 84.4 | 1.1 | 35 | 10 | 0.12 | 59 | 0.08 |
| Example 3 | | | | | | | | | | |

TABLE 2-1

| | | | | | | | | | | | Amount of | Amount of | | Calcination |
|---|---|---|---|---|---|---|---|---|---|---|---|---|---|---|
| | | | | | | | | | 12 nm | 41 nm | oxalic | 16.6 wt % | First | temperature |
| | | | | | | | | | silica | silica | acid | nitric acid | Mixture | for catalyst |
| | Mo | Bi | Fe | Co | Ni | Mg | Ce | Rb | sol wt % | sol wt % | added (g) | added (g) | pH | (° C.) |
| Example 5 | 12.00 | 0.34 | 1.58 | 4.38 | 3.11 | 0 | 0.89 | 0.11 | 50 | 50 | 25 | 249 | 4.3 | 590 |
| Example 6 | 12.00 | 0.34 | 1.58 | 4.38 | 3.11 | 0 | 0.89 | 0.11 | 50 | 50 | 25 | 190 | 4.5 | 590 |
| Example 7 | 12.00 | 0.34 | 1.58 | 4.38 | 3.11 | 0 | 0.89 | 0.11 | 50 | 50 | 25 | 158 | 4.8 | 590 |
| Comparative | 12.00 | 0.34 | 1.58 | 4.38 | 3.11 | 0 | 0.89 | 0.11 | 50 | 50 | 25 | 299 | 3.8 | 590 |
| Example 4 | | | | | | | | | | | | | | |
| Comparative | 12.00 | 0.34 | 1.58 | 4.38 | 3.11 | 0 | 0.89 | 0.11 | 50 | 50 | 25 | 0 | 5.8 | 590 |
| Example 5 | | | | | | | | | | | | | | |

TABLE 2-2

| | | | | | | Specific | 0 to 30 | 0 to 30 | 30 to 1,000 | 30 to 1,000 |
|---|---|---|---|---|---|---|---|---|---|---|
| | A: Si atom | B: Si atom | | AN | Wear | surface | nm modal | nm pore | nm modal | nm pore |
| | on particle | in entire | Ratio | yield | loss | area | diameter | volume | diameter | volume |
| | surface wt % | particle wt % | A/B | (%) | (%) | (m²/g) | (nm) | (ml/g) | (nm) | (ml/g) |
| Example 5 | 0.295 | 0.305 | 0.970 | 85.1 | 1.3 | 32 | 15 | 0.12 | 68 | 0.10 |
| Example 6 | 0.305 | 0.305 | 1.000 | 85.0 | 1.3 | 30 | 14 | 0.11 | 65 | 0.11 |
| Example 7 | 0.312 | 0.305 | 1.024 | 84.8 | 1.1 | 30 | 14 | 0.12 | 65 | 0.11 |
| Comparative | 0.268 | 0.305 | 0.880 | 84.2 | 3.7 | 31 | 15 | 0.12 | 68 | 0.11 |
| Example 4 | | | | | | | | | | |
| Comparative | 0.329 | 0.305 | 1.080 | 84.6 | 1.0 | 31 | 14 | 0.12 | 63 | 0.11 |
| Example 5 | | | | | | | | | | |

TABLE 3-1

| | | | | | | | | | | | Amount of | Amount of | | Calcination |
|---|---|---|---|---|---|---|---|---|---|---|---|---|---|---|
| | | | | | | | | | 12 nm | 41 nm | oxalic | 16.6 wt % | First | temperature |
| | | | | | | | | | silica | silica | acid | nitric acid | mixture | for catalyst |
| | Mo | Bi | Fe | Co | Ni | Mg | Ce | Rb | sol wt % | sol wt % | added (g) | added (g) | pH | (° C.) |
| Example 8 | 12.00 | 0.34 | 1.58 | 4.38 | 3.11 | 0 | 0.89 | 0.11 | 100 | 0 | 25 | 250 | 4.2 | 610 |
| Example 9 | 12.00 | 0.34 | 1.58 | 4.38 | 3.11 | 0 | 0.89 | 0.11 | 100 | 0 | 25 | 189 | 4.6 | 610 |
| Example 10 | 12.00 | 0.34 | 1.58 | 4.38 | 3.11 | 0 | 0.89 | 0.11 | 100 | 0 | 25 | 160 | 4.8 | 610 |
| Comparative | 12.00 | 0.34 | 1.58 | 4.38 | 3.11 | 0 | 0.89 | 0.11 | 100 | 0 | 25 | 296 | 3.9 | 610 |
| Example 6 | | | | | | | | | | | | | | |
| Comparative | 12.00 | 0.34 | 1.58 | 4.38 | 3.11 | 0 | 0.89 | 0.11 | 100 | 0 | 25 | 0 | 5.6 | 610 |
| Example 7 | | | | | | | | | | | | | | |

TABLE 3-2

| | | | | | | Specific | 0 to 30 | 0 to 30 | 30 to 1,000 | 30 to 1,000 |
|---|---|---|---|---|---|---|---|---|---|---|
| | A: Si atom | B: Si atom | | AN | Wear | surface | nm modal | nm pore | nm modal | nm pore |
| | on particle | in entire | Ratio | yield | loss | area | diameter | volume | diameter | volume |
| | surface wt % | particle wt % | A/B | (%) | (%) | (m²/g) | (nm) | (ml/g) | (nm) | (ml/g) |
| Example 8 | 0.292 | 0.305 | 0.960 | 84.8 | 0.5 | 38 | 10 | 0.10 | 50 | 0.08 |
| Example 9 | 0.308 | 0.305 | 1.010 | 84.7 | 0.4 | 40 | 10 | 0.11 | 51 | 0.07 |
| Example 10 | 0.314 | 0.305 | 1.030 | 84.5 | 0.4 | 41 | 10 | 0.10 | 50 | 0.07 |
| Comparative | 0.284 | 0.305 | 0.932 | 83.7 | 1.7 | 40 | 12 | 0.11 | 54 | 0.08 |
| Example 6 | | | | | | | | | | |
| Comparative | 0.337 | 0.305 | 1.105 | 84.2 | 0.3 | 39 | 10 | 0.10 | 52 | 0.07 |
| Example 7 | | | | | | | | | | |

TABLE 4-1

| | | | | | | | | | | | Amount | Amount of | | Calcination |
|---|---|---|---|---|---|---|---|---|---|---|---|---|---|---|
| | | | | | | | | | 12 nm | 41 nm | of oxalic | 16.6 wt % | First | temperature |
| | | | | | | | | | silica | silica | acid | nitric acid | mixture | for catalyst |
| | Mo | Bi | Fe | Co | Ni | Mg | Ce | Rb | sol wt % | sol wt % | added (g) | added (g) | pH | (° C.) |
| Example 11 | 12.00 | 0.34 | 1.48 | 0 | 6.35 | 1.70 | 0.53 | 0.17 | 80 | 20 | 0 | 250 | 4.2 | 580 |
| Example 12 | 12.00 | 0.34 | 1.48 | 0 | 6.35 | 1.70 | 0.53 | 0.17 | 80 | 20 | 0 | 191 | 4.5 | 580 |
| Example 13 | 12.00 | 0.34 | 1.48 | 0 | 6.35 | 1.70 | 0.53 | 0.17 | 80 | 20 | 0 | 160 | 4.8 | 580 |
| Example 14 | 12.00 | 0.34 | 1.48 | 0 | 6.35 | 1.70 | 0.53 | 0.17 | 80 | 20 | 25 | 250 | 4.1 | 580 |
| Comparative | 12.00 | 0.34 | 1.48 | 0 | 6.35 | 1.70 | 0.53 | 0.17 | 80 | 20 | 0 | 295 | 3.9 | 580 |
| Example 8 | | | | | | | | | | | | | | |
| Comparative | 12.00 | 0.34 | 1.48 | 0 | 6.35 | 1.70 | 0.53 | 0.17 | 80 | 20 | 0 | 0 | 5.9 | 580 |
| Example 9 | | | | | | | | | | | | | | |
| Comparative | 12.00 | 0.34 | 1.48 | 0 | 6.35 | 1.70 | 0.53 | 0.17 | 80 | 20 | 25 | 295 | 3.7 | 580 |
| Example 10 | | | | | | | | | | | | | | |

TABLE 4-2

| | | | | | | Specific | 0 to 30 | 0 to 30 | 30 to 1,000 | 30 to 1,000 |
|---|---|---|---|---|---|---|---|---|---|---|
| | A: Si atom | B: Si atom | | AN | Wear | surface | nm modal | nm pore | nm modal | nm pore |
| | on particle | in entire | Ratio | yield | loss | area | diameter | volume | diameter | volume |
| | surface wt % | particle wt % | A/B | (%) | (%) | (m²/g) | (nm) | (ml/g) | (nm) | (ml/g) |
| Example 11 | 0.304 | 0.307 | 0.990 | 83.5 | 0.8 | 36 | 11 | 0.12 | 59 | 0.07 |
| Example 12 | 0.313 | 0.307 | 1.020 | 83.4 | 0.8 | 36 | 11 | 0.12 | 58 | 0.08 |
| Example 13 | 0.316 | 0.307 | 1.030 | 83.3 | 0.6 | 35 | 11 | 0.12 | 58 | 0.08 |
| Example 14 | 0.293 | 0.307 | 0.955 | 83.8 | 0.9 | 36 | 11 | 0.12 | 59 | 0.07 |
| Comparative | 0.275 | 0.307 | 0.895 | 82.5 | 2.3 | 36 | 11 | 0.12 | 58 | 0.07 |
| Example 8 | | | | | | | | | | |
| Comparative | 0.345 | 0.307 | 1.124 | 83.0 | 0.6 | 35 | 12 | 0.12 | 56 | 0.07 |
| Example 9 | | | | | | | | | | |
| Comparative | 0.267 | 0.307 | 0.870 | 82.2 | 2.8 | 35 | 12 | 0.12 | 56 | 0.08 |
| Example 10 | | | | | | | | | | |

1. A catalyst comprising a metal oxide, which is an oxide of a metal including molybdenum, bismuth and iron, and silica supporting the metal oxide,
wherein when a surface parameter of silicon by SEM-EDX is A and a bulk parameter of silicon by XRF is B, A/B is 0.95 to 1.04.

2. The catalyst according to claim 1, wherein a mass ratio of the silica to the metal oxide is 7:3 to 2:8.

3. The catalyst according to claim 1, wherein A/B is 0.95 to 1.02.

4. The catalyst according to claim 1, which is used for a vapor-phase catalytic ammoxidation reaction.

5. The catalyst according to claim 1, wherein the metal oxide is represented by the following formula (1): 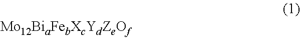
wherein
X is at least one selected from the group consisting of nickel, cobalt, magnesium, calcium, zinc, strontium and barium,
Y is at least one selected from the group consisting of cerium, chromium, lanthanum, neodymium, yttrium, praseodymium, samarium, gallium and indium,
Z is at least one selected from the group consisting of sodium, potassium, rubidium and cesium,
a is 0.1 or more and 2.0 or less,
b is 0.1 or more and 2.8 or less,
c is 0.1 or more and 10.0 or less,
d is 0.1 or more and 3.0 or less,
e is 0.01 or more and 2.0 or less, and
f is a value determined by X to Z and a to e.

6. The catalyst according to claim 1, wherein the catalyst has a specific surface area of 10 to 50 m²/g.

7. The catalyst according to claim 1, wherein
in a pore distribution of the catalyst,
the catalyst has a first modal diameter being within a range of 5 to 25 nm in a first range in which the pore diameter is 0 nm or more and less than 30 nm,
the catalyst has a second modal diameter being within a range of 35 to 200 nm in a second range in which the pore diameter is 30 nm or more and 1,000 nm or less,
the catalyst has a total volume of the pores of 0.05 cm³/g or more in the first range, and
the catalyst has a total volume of the pores of 0.05 cm³/g or more in the second range.

8. A method for producing a catalyst, comprising:
a step of providing a first mixture which comprises a silica starting material and molybdenum and has a pH of 4.1 to 5.5,
a step of preparing a second mixture by mixing the first mixture with bismuth and iron,
a step of preparing particles by spray-drying the second mixture, and
a step of preparing catalyst by calcining the particles.

9. The method according to claim 8, wherein the pH of the first mixture is adjusted to 4.1 to 5.5 by ammonia water or nitric acid.

10. The method according to claim 8, wherein the silica starting material is silica sol and the silica sol has a median diameter of 50 nm or less.

11. A method for producing acrylonitrile, comprising:
a step of preparing acrylonitrile by reacting propylene, ammonia and molecular oxygen in the presence of the catalyst according to claim 1.

12. The method according to claim 11, wherein the reaction is performed in a fluidized bed reactor.
